(19) United States
(12) Patent Application Publication
(10) Pub. No.: US 2007/0298107 A1
(43) Pub. Date: Dec. 27, 2007

(54) Pharmaceutical Compositions of Mirtazapine

(75) Inventors: Phanindrudu Aluri, Hyderabad (IN); Justin Babu, Hyderabad (IN); Shaik Srinivasa Rao, Hyderabad (IN); Ashish Gogia, Hyderabad (IN)

Correspondence Address: Jay Akhave, 2058 N. Mills Ave. #612, Claremont, CA, 91711, US

(73) Assignee: AUROBINDO PHARMA LTD., Hyderabad (IN)

(21) Appl. No.: 10/580,391

(22) PCT Filed: Nov. 24, 2004
(86) PCT No.: PCT/IB04/03872

§ 371 Date: Jun. 4, 2007

(30) Foreign Application Priority Data
Nov. 25, 2003 (IN) 964/CHE/2003
Sep. 17, 2004 (IN) 930/CHE/2004

(51) Int. Cl. A61K 9/20; A61K 31/55; A61K 9/28; A61P 25/24

(52) U.S. Cl. 424/474; 424/464; 514/214.02

(57) ABSTRACT

The present invention relates to pharmaceutical compositions of mirtazapine or its pharmaceutically acceptable salts. More particularly, the present invention relates to solid unit dosage forms of anhydrous mirtazapine or its pharmaceutically acceptable salts suitable for oral administration. The present invention also relates to a process for the preparation of pharmaceutical compositions of mirtazapine or its pharmaceutically acceptable salts.

### FIELD OF THE INVENTION

The present invention relates to pharmaceutical compositions of mirtazapine or its pharmaceutically acceptable salts. More particularly, the present invention relates to solid unit dosage forms of anhydrous mirtazapine or its pharmaceutically acceptable salts suitable for oral administration.

The present invention also relates to a process for the preparation of pharmaceutical compositions of mirtazapine or its pharmaceutically acceptable salts.

### BACKGROUND OF THE INVENTION

Mirtazapine is disclosed and claimed in U.S. Pat. No. 4,062,848. Mirtazapine, is approved, under the trademark REMERON and REMERON SOLTAB by the US Food and Drug Administration, for the treatment of depression. Mirtazapine has a tetra cyclic chemical structure unrelated to other classes of antidepressants such as selective serotonin reuptake inhibitors, tricyclics or monoamine oxidase inhibitors.

Mirtazapine is conventionally being marketed as mirtazapine hemihydrate. The usual practice to produce conventional tablets with mirtazapine hemihydrate is to micronize the active ingredient to produce 90% of the particles <100 microns and mix it with excipients and compress it to produce tablets. Whereas the process for producing mirtazapine orally disintegrable tablets involves the use of effervescent or non-direct compressible filler (low particle size) and mix with micronized mirtazapine hemihydrate and compressed to get mirtazapine ODT.

U.S. Pat. Nos. 6,375,982 and 6,589,556 disclose a rapid melt, semi-solid molded composition comprising: at least one binder in an amount from about 0.01% to about 70% by weight; a salivating agent in an amount from about 0.05% to about 15% by weight; a diluent/bulking material in an amount from about 10% to about 90% by weight; and a therapeutically effective amount of a drug.

U.S. Pat. Nos. 4,371,516, 5,501,816 and 5,720,974 disclose processes for the preparation of porous, rapidly disintegrable tablets, which include the steps of adding a small quantity of a solvent to sugars, alcohols or carbohydrates to obtain a tablet mixture and removing the solvent therefrom. However, these processes have low productivity due to the involvement of complicated process steps and the tablets obtained thereby are easily friable and do not meet the hardness required for withstanding breakage during commercial handling.

WO 01/26621 discloses a pharmaceutical formulation of mirtazapine and pharmaceutically acceptable excipients, characterized in that the dosage unit is of the orally disintegrating type, and the formulation comprises means which substantially prevent mirtazapine from being released orally and the means which substantially prevent mirtazapine from being released orally is a polymer layer coating mirtazapine.

Cima Labs has produced oral dosage forms including microparticles and effervescents which rapidly disintegrate in the mouth and provide adequate taste-masking (U.S. Pat. No. 5,178,878). Zydis, on the other hand, produces a rapidly dissolvable, freeze-dried, sugar matrix to produce a rapidly dissolving tablet. While these dosage forms are effective, they provide significant problems in terms of production, storage, transport and during consumer usage. They are also significantly more costly to produce.

Mirtazapine is essentially insoluble in water. The Particle Size Distribution (PSD) of mirtazapine crystals are used to determine the available surface area for the drug dissolution, thus effecting the solubility. Often, it is observed that the available surface area for drug dissolution correlates to the rate of dissolution and solubility where a greater surface area enhances the solubility of a drug and enhances the rate of dissolution of a drug. Further, the velocity of dissolution of a drug often effects the drug's bioavailability. Thus the PSD of mirtazapine and, in particular, the mean particle diameter are important parameters to characterize and predict the bioavailability of the drug. It is desirable to have mirtazapine with a particle size in which the mean particle size enhances the reproducibility of the rate of dissolution and the reproducibility of the dissolution. It is desirable to have mirtazapine in which the mean particle size imparts an improved and stable dissolution profile.

Freeze drying processes have been used to prepare fast disintegrating dosage forms. Although this technology produces a product which rapidly disintegrates in water or in the oral cavity, a drawback is represented by the poor physical integrity of its physical structure which severely limits further manufacturing operations such as forming blister packs. Another significant drawback of the freeze drying technology in manufacturing such dosage forms is the high production costs because of the lengthy duration of each freeze drying cycle (normally from 24 to 48 hours).

The process micronization is tedious and hazardous, since it involves milling and exposure of drug dust to operating personnel. Mirtazapine being an antidepressant, too much exposure of the drug may pose health problems.

The inventors of the present invention have surprisingly found that the use of anhydrous mirtazapine avoids the process of micronization and the handling of anhydrous mirtazapine is safe and user friendly. Although the present invention uses the new form of mirtazapine i.e. anhydrous mirtazapine, yet when formulated gives both ODT and conventional tablets meeting the USP standards as well as other in vitro and in vivo results matching the reference product.

### OBJECTIVE OF THE INVENTION

Accordingly, the main objective of present invention is to provide compositions for anhydrous mirtazapine in such a way that it will comply with the reference product in terms of in vivo parameters like bioequivalence and in vitro parameters like dissolution, disintegration and etc.

Another objective of the present invention is to provide simple, cost effective and efficient process for preparing the solid dosage forms of mirtazapine on a commercial scale with adequate hardness and good reproducibility.

Yet another objective of the present invention is to provide film-coated tablets of anhydrous mirtazapine.

Yet another objective of the present invention is to provide a hard, compressed, orally disintegrable dosage form of anhydrous mirtazapine.

### SUMMARY OF THE INVENTION

According to an embodiment of the present invention, there is provided unit dosage forms of anhydrous mirtazapine or its pharmaceutically acceptable salts suitable for oral administration.

In yet another embodiment of the present invention, there is provided film-coated tablets of mirtazapine which comprises anhydrous mirtazapine or its pharmaceutically acceptable salts, low-substituted hydroxypropylcellulose and one or more pharmaceutically acceptable excipients.

In yet another embodiment of the present invention, there is provided a hard, compressed, orally disintegrable tablet dosage form of anhydrous mirtazapine comprising mirtazapine or its pharmaceutically acceptable salts, and one or more non-effervescent excipients.

In yet another embodiment of the present invention, there is provided a process for the preparation of film-coated tablets of anhydrous mirtazapine.

In yet another embodiment of the present invention, there is provided a process for the preparation of hard, compressed, orally disintegrable tablet dosage form of anhydrous mirtazapine.

### DETAILED DESCRIPTION OF THE INVENTION

The present invention involves the use of anhydrous mirtazapine having coarser particle size, which avoids the use of air jet milling to reduce the particle size to <100 microns and thereby reduces the length of the unit process. The other advantage of using anhydrous mirtazapine is its ease of handling.

In yet another embodiment of the present invention, there is provided unit dosage forms of anhydrous mirtazapine having a particle size distribution (PSD) of 90% particles less than 600 μm, more preferably the particle size distribution (PSD) for the above formulation process of mirtazapine is 90% particles less than 400 μm.

The present invention uses substantially simple and cost effective manufacturing technique.

The amount of mirtazapine according to the composition of the present invention is in the range of 1 to 100 mg.

The term non-effervescent excipients as used in orally disintegrating compressed tablets comprise binders, dispersing agents, fillers, flavoring agents, sweetening agents, lubricants or glidants and the like.

The term pharmaceutically acceptable excipients as used in film coated tablet comprise binders, dispersing agents, fillers, lubricants or glidants and the like.

In yet another embodiment of the present invention, the orally disintegrating compressed tablets comprises anhydrous mirtazapine from about 1 to 50%, and a mixture of non-effervescent excipients comprising from about 10% to 80% of one or more diluents, at least one dispersing agent in an amount of 2% to 15%, from 0% to 15% of one or more binders.

In yet another embodiment of the present invention, there is provided orally disintegrated tablets of anhydrous mirtazapine, which disintegrates within 3 minutes, preferably within 60 seconds, more preferably within 30 seconds.

The dispersing agent used in accordance with the present invention is selected from crosscarmellose sodium, crosspovidone, sodium starch glycolate, sodium carboxymethyl cellulose, hydroxypropyl cellulose, xanthan gum, alginic acid, alginates, carbopols and the like or combination thereof, preferably dispersing agent used is crosspovidone.

The diluents used according to the present invention are selected from calcium phosphate-dibasic, cellulose-microcrystalline, cellulose powdered, calcium silicate, ployols such as mannitol, sorbitol, xylitol, maltitol, sucrose and combinations thereof.

Suitable binders according to the present invention are selected from methylcellulose, hydroxypropyl cellulose, hydroxypropyl methylcellulose, polyvinylpyrrolidone, gelatin, gum arabic, ethyl cellulose, polyvinyl alcohol, pullulan, starch, pregelatinized starch, agar, tragacanth, sodium alginate, propylene glycol, alginate, plasdone and the like.

Suitable lubricants according to the present invention are selected from talc, magnesium stearate, stearic acid or glyceryl behenate, preferably magnesium stearate and suitable glidants include colloidal silicon dioxide or talc, preferably colloidal silicon dioxide.

Suitable sweeteners according to the present invention is selected from sugars such as sucrose, lactose and glucose; saccharin and salts thereof; mannitol and aspartame.

Suitable flavoring agents include strawberry guarana, peppermint, cherry, mint, caramel, raspberry, lemon, orange, tuttifruity, banana, bubble gum, preferably strawberry guarana, peppermint flavor or combination thereof.

Owing to certain handling advantages of particle size distribution (PSD) of mirtazapine, the formulation process of the anhydrous mirtazapine with some suitable adjuvants is contemplated and achieved in our lab with satisfactory results in terms of the formulation parameters which constitute both in vitro as well as in vivo aspects.

The present formulation process for preparing the solid dosage forms of anhydrous mirtazapine led to unexpected results of possessing a more stable and reproducible dissolution profile. When compared to mirtazapine made by conventional methods, the present formulation process of anhydrous mirtazapine unexpectedly demonstrated a more reproducible dissolution curve and a smaller standard deviation. This valuable improvement provides for more accurate dosing of mirtazapine.

The different formulation processes that can be employed for making the disclosed formulations are dry granulation, wet granulation, slugging, compaction and direct compression. But preferably, the tablets of the present invention are prepared by wet granulation technique.

The formulation process for the preparation of film coated tablets according to the present invention are carried out by sifting the ingredients, blending anhydrous mirtazapine with disintegrants, diluents and/or binders that are intended to be used; milling and then granulating the blend; drying the granules and sifting to get the desired size; mixing the dried granules with rest of the diluents, lubricants and compressing the blend to form tablets and coating the tablets using conventional coating techniques.

The formulation process for the preparation of orally disintegrable tablet dosage form of mirtazapine according to the present invention are carried out by sifting the ingredients, blending anhydrous mirtazapine with disintegrants, diluents and/or binders that are intended to be used; milling the sifted materials; granulating the blend with the solvent; drying the granules and sifting to get the desired size; mixing the dried granules with rest of the diluents, lubricants, flavoring agents, sweetening agents, and compressing the blend to form tablets.

Alternatively, the process may also involve the preparation of placebo granules as per the procedure explained in the above paragraph and adding the active ingredient during the lubrication stage.

The following examples further exemplify the inventions and are not intended to limit the scope of the inventions. It is obvious to those skilled in the art to find out the composition for other dosage forms and substitute the equivalent excipients as described in this specification or with the one known to the industry.

### EXAMPLE 1

Formulation of film coated tablets of mirtazapine

| Ingredients | Quantity mg/tablet |
|---|---|
| Mirtazapine (Anhydrous) | Equivalent to Mirtazapine |
| (90% of particles less than 400 Microns) | 7.5/15/30/45 mg |
| Lactose monohydrate | 63.00-73.00% |
| Hydroxypropyl cellulose | 1.5-3.5% |
| Starch | 6.5-8.5% |
| Colloidal silicon dioxide | 0.54-1.54% |
| Low-substituted hydroxypropyl cellulose | 6.5-8.5% |
| Magnesium stearate | 0.26-0.66% |
| Opadry yellow | 2.0-4.0% |
| Purified water | q.s |

The processing steps that are involved in making the film coated tablets of mirtazapine disclosed above are given below:

i) Sifted anhydrous mirtazapine through 425 μm sieve, hydroxypropyl cellulose and lactose monohydrate through 850μ sieve separately,

ii) resifted mirtazapine and hydroxypropyl cellulose together through 850μ sieve,

iii) milled the material of step (ii),

iv) rinsed the mill with sufficient quantity of sifted lactose,

v) mixed the material of step (iii) and lactose monohydrate in a rapid mixer granulator and granulated with purified water,

vi) dried the granules in a fluid bed dryer,

vii) sifted the dried granules through 850 μm sieve and collected the retentions separately,

viii) milled the retentions in a multi mill,

ix) sifted the milled granules of through 850μ sieve,

x) sifted the extra granular low substituted hydroxypropyl cellulose through 850μ sieve and sifted starch and anhydrous colloidal silicon dioxide together through 850μ,

xi) mixed the sifted low substituted hydroxypropyl cellulose, starch and colloidal silicon dioxide from step (x) with the material of step (ix),

xii) lubricated the material of step (xi) with sifted magnesium stearate,

xiii) compressed the lubricated blend into tablets and

xiv) prepared Opadry coating suspension in water and coated the tablets of step (xiii) with coating suspension till the desired weight build-up is achieved and dried the coated tablets.

Examples 2-4 represents orally disintegrating tablets of anhydrous mirtazapine. The processing steps that are involved in making orally disintegrating tablets of anhydrous mirtazapine as disclosed in examples 2-4 are given below:

i) sifted mirtazapine, half the quantity of dispersing agent, binder, diluent through 425 μm mesh,

ii) milled the sifted material of step (i) through multimill,

iii) loaded the materials of step (ii) in a rapid mixer granulator and mixed for 15 minutes with impeller at slow speed,

iv) added purified water over a period of 2-3 minutes with impeller at slow speed

v) kneaded the wet mass for 1 minute with only impeller followed by both impeller and chopper at slow speed for 1 min,

vi) dried the wet mass of step (v) at an inlet temperature of 60° C.±5° C. in fluid bed drier,

vii) sifted the dried granules of step vi through 600 μm mesh and milled the retentions using multimill with 1.5 mm screen at slow speed/knives forward,

viii) sifted the milled material of step (vii) through 600 μm mesh,

ix) sifted the remaining half of dispersing agent, diluents through 425 μm mesh,

x) sifted flavoring agents, sweeteners, glidants, lubricant through 425 μm mesh,

xi) loaded the granules of step (viii) in low shear blender,

xii) loaded sifted material of step (ix) and (x) except lubricant and blend for 10 minutes,

xiii) added sifted lubricant in to the low shear blender and blend for 5 minutes,

xiv) compressed the lubricated blend to obtain mirtazapine orally disintegrating tablets.

### EXAMPLE 2

| Ingredients | Quantity mg/tablet |
|---|---|
| Mirtazapine (Anhydrous) | 30.00 |
| Crospovidone USNF | 20.00 |
| Mannitol USP | 40.00 |
| Microcrystalline cellulose USNF | 98.00 |
| Aspartame USNF | 6.00 |
| Strawberry guarana flavor IH | 1.60 |
| Peppermint flavor IH | 0.40 |
| Colloidal silicon dioxide USNF | 2.00 |
| Magnesium stearate USNF | 2.00 |
| Purified water IH | q.s |

### EXAMPLE 3

| Ingredients | Quantity mg/tablet |
|---|---|
| Mirtazapine (Anhydrous) | 15.00 |
| Crospovidone | 10.00 |
| Mannitol | 20.00 |
| Pregelatinised Starch | 5.00 |
| Microcrystalline cellulose | 46.00 |
| Aspartame | 1.00 |
| Orange flavor | 1.00 |
| Colloidal silicon dioxide | 1.00 |
| Magnesium stearate | 1.00 |
| Purified water | q.s |

### EXAMPLE 4

| Ingredients | Quantity mg/tablet |
|---|---|
| Mirtazapine (Anhydrous) | 15.00 |
| Crospovidone | 5.00 |
| Mannitol | 20.00 |
| Calcium Silicate | 10.00 |
| Microcrystalline cellulose | 44.00 |
| Aspartame | 3.00 |
| Strawberry guarana flavor | 0.80 |
| Peppermint flavor | 0.20 |
| Colloidal silicon dioxide | 1.00 |
| Magnesium stearate | 1.00 |
| Purified water | q.s |

We claim:

1. A dosage form comprising anhydrous mirtazapine or its pharmaceutically acceptable salts.

2. The dosage form as claimed in claim 1, wherein the dosage form comprises film-coated tablets of anhydrous mirtazapine or its pharmaceutically acceptable salts, low-substituted hydroxypropylcellulose and one or more pharmaceutically acceptable excipients.

3. The dosage form as claimed in claim 1, wherein the dosage form comprises orally disintegrable tablets of anhydrous mirtazapine or its pharmaceutically acceptable salts, and one or more non-effervescent excipients.

4. The dosage form as claimed in claim 2, wherein the particle size distribution of anhydrous mirtazapine or its pharmaceutically acceptable salt used in the tablet is such that the diameter of 90% of the particles is less than 600 μm, more preferably less than 400 μm.

5. The dosage form as claimed in claim 3, wherein the particle size distribution of anhydrous mirtazapine or its pharmaceutically acceptable salt used in the tablet is such that the diameter of 90% of the particles is less than 600 μm, more preferably less than 400 μm.

6. A process for the preparation of film-coated tablets of mirtazapine, comprising anhydrous mirtazapine or its pharmaceutically acceptable salts, low substituted hydroxypropylcellulose and one or more pharmaceutically acceptable excipients.

7. A process for the preparation of hard, compressed, orally disintegrable tablet dosage form of mirtazapine comprising anhydrous mirtazapine or its pharmaceutically acceptable salts, and one or more non-effervescent excipients.

8. The dosage form as claimed in claim 3, wherein the said non-effervescent excipients comprise binders, diluents, dispersing agents, flavoring agents, sweetening agents, lubricants, glidants.

9. The dosage form as claimed in claim 3, further comprises anhydrous mirtazapine or its pharmaceutically acceptable salt from about 1 to 50% by weight of the tablet.

10. The dosage form as claimed in claim 8, wherein the dispersing agent is selected from the group consisting of crosscarmellose sodium, crosspovidone, sodium starch glycolate, sodium carboxymethyl cellulose, hydroxypropyl cellulose, xanthan gum, alginic acid, alginates and carbopols and combination thereof.

11. The dosage form as claimed in claim 8, wherein the diluent is selected from the group consisting of calcium phosphate-dibasic, cellulose-microcrystalline, cellulose powdered, calcium silicate, ployols such as mannitol, sorbitol, xylitol, maltitol, sucrose, lactose and combinations thereof.

12. The dosage from as claimed in claim 8, where the binder is selected from the group consisting of methylcellulose, hydroxypropyl cellulose, hydroxypropyl methylcellulose, polyvinylpyrrolidone, gelatin, gum Arabic, ethyl cellulose, polyvinyl alcohol, pullulan, starch, pregelatinized starch, agar, tragacanth, sodium alginate, propylene glycol, alginate, plasdone and combinations thereof.

13. The dosage from as claimed in claim 8, wherein the lubricant is selected from the group consisting of talc, magnesium stearate, stearic acid, glyceryl behenate and mixtures thereof and glidants is selected from the group consisting of colloidal silicon dioxide, talc and mixtures thereof.

14. The dosage form as claimed in claim 8, wherein the sweetener is selected from the group consisting of sugars, sucrose, lactose, glucose, saccharin, saccharin salts, mannitol, aspartame and combinations thereof.

15. The dosage form as claimed in claim 8, wherein the flavoring agent is selected from the group consisting of strawberry guarana, peppermint, cherry, mint, caramel, raspberry, lemon, orange, tuttifruity, banana, bubble gum, preferably strawberry, guarana, peppermint flavor or combination thereof.

16. The dosage form as claimed in claim 2, wherein the pharmaceutically acceptable excipients comprise binders, diluents, dispersing agents, lubricants and glidants.

17. The dosage form as claimed in claim 16, wherein the dispersing agent is selected from the group consisting of crosscarmellose sodium, crosspovidone, sodium starch glycolate, sodium carboxymethyl cellulose, hydroxypropyl cellulose, xanthan gum, alginic acid, alginates, and carbopols and mixtures thereof.

18. The dosage form as claimed in claim 16, wherein the diluent is selected from the group consisting of calcium phosphate-dibasic, cellulose-microcrystalline, cellulose powdered, calcium silicate, polyols, mannitol, sorbitol, xylitol, maltitol, sucrose and combinations thereof.

19. The dosage form as claimed in claim 16, wherein the binder is selected from the group consisting of methylcellulose, hydroxypropyl cellulose, hydroxypropyl methylcellulose, polyvinylpyrrolidone, gelatin, gum arabic, ethyl cellulose, polyvinyl alcohol, pullulan, starch, pregelatinized starch, agar, tragacanth, sodium alginate, propylene glycol, alginate, plasdone and mixtures thereof.

20. (canceled)

21. (canceled)

22. A process for the preparation of film coated tablets of anhydrous mirtazapine or its pharmaceutically acceptable salts comprising the steps of:
i) blending anhydrous mirtazapine with disintegrants, diluents and/or binders
ii) milling and granulating the blend with purified water to obtain granules,
iii) drying the said granules and mixing the dried granules with diluents and lubricants,
iv) compressing the granule mixture into tablets,
v) coating the tablets.

23. A process for the preparation of orally disintegrating tablets of anhydrous mirtazapine or its pharmaceutically acceptable salts comprising the steps of:
i) blending anhydrous mirtazapine with disintegrants, diluents and/or binders,
ii) milling and granulating the blend with a solvent to obtain granules,
iii) drying the said granules and mixing the dried granules with diluents, lubricants, flavoring agents, sweetening agents,
iv) compressing the granule mixture into tablets.
